(19) United States
(12) Patent Application Publication
(10) Pub. No.: US 2021/0293819 A1
(43) Pub. Date: Sep. 23, 2021

(54) METHOD FOR IDENTIFYING CANCER-SPECIFIC ANTIBODIES UTILIZING IMMUNE CHECKPOINT INHIBITION

(71) Applicant: Elliot Davis, Longwood, FL (US)

(72) Inventor: Elliot Davis, Longwood, FL (US)

(21) Appl. No.: 16/822,307

(22) Filed: Mar. 18, 2020

(51) Int. Cl. G01N 33/574; C07K 16/30; A61K 35/17

(52) U.S. Cl. CPC G01N 33/574; A61K 35/17; C07K 16/30

(57) ABSTRACT

Inhibition of immune checkpoints in cancer patients may induce the patients B cells to generate antibodies against their cancer cells. The present disclosure provides methods for isolating, identifying, and characterizing these cancer-specific antibodies, and/or their antibodies/antigen binding sites. In particular, the presently disclosed methods relate to isolating, identifying, and characterizing cancer-specific antibodies from cancer patients in conjunction with an acute treatment of checkpoint inhibitor/s, and methods of generating cancer-specific medications thereof.

### TECHNICAL FIELD

The presently disclosed subject matter relates generally to a method for identifying cancer-specific antibodies utilizing immune checkpoint inhibition.

### BACKGROUND

Immune checkpoints are a normal part of the immune system. Generally, their role is to prevent an immune response from being too strong and destroying healthy cells in the body. As part of an effort to harness a cancer patient's own immune system to fight his/her cancer, immunotherapy drugs that belong to a category of immune checkpoint inhibitors have been developed. Those drugs generally work by blocking checkpoint proteins, thus preventing an inhibition of the immune response to cancer cells. For example, administration of immune checkpoint inhibitors may allow T cells to kill cancer cells. Inhibition of immune checkpoints in cancer patients may also result in inducing the cancer patient's B cells to generate antibodies against the cancer cells.

Immune checkpoint inhibitors may be are less toxic and easier to tolerate than most chemotherapy drugs, and they are already in use with a wide range of cancer types. The present disclosure provides a method for utilizing immune checkpoint inhibitors for isolation and characterization of cancer-specific antibodies from the patient being treated.

### SUMMARY

In accordance with the present invention, various embodiments of utilizing immune checkpoint inhibitors for isolation and characterization of cancer-specific antibodies and methods of generation of cancer medication/s thereof are disclosed. In one embodiment, the present disclosure provides a method for identifying cancer-specific antibodies utilizing an immune checkpoint inhibition treatment in a subject who has cancer comprising the steps of:
a. obtaining cancer cells and/or their components, and normal cells and/or their components from the subject who has cancer;
b. administering the immune checkpoint inhibition treatment to the subject who has cancer;
c. allowing time for generation of an immune reaction against the cancer cells in the subject who has cancer;
d. obtaining a serum sample from subject who has cancer, the sample comprising any antibodies which may have been generated against the cancer cells;
e. optionally discontinuing the immune checkpoint inhibition treatment of the subject who has cancer;
f. removing from the serum of step d antibodies that bind to the normal cells and/or components by incubating the serum with a cell culture prepared of the normal cells and/or their components of step a, allowing sufficient time for antibodies/antigens binding and generation of antibodies/antigen complexes, and removing and keeping the supernatant;
g. selecting the antibodies that bind to the cancer cells and/or their components by incubating the supernatant of step f with a culture of the cancer cells and/or their components of step a, allowing sufficient time for antibodies/antigens binding and generation of antibodies/antigen complexes, keeping the culture comprising antibodies/antigen complexes for further analysis and discarding the supernatant;
h. extracting the antibody/antigen complexes from the culture of step g;
i. separating the antibodies and the antigens of the antibody/antigen complexes of step h using standard protocols;
j . analyzing the antigens of step i are using standard methods for antigen identification, such as mass spectrometry; and,
k. identifying sites or regions on the antigens of step j which are specific to the cancer cells or their components obtained from the subject who has cancer in step a.

In some embodiments, the presently disclosed method comprises using the antigens of step j, or the sites or regions, identified in step k to produce a medication, wherein the medication may be antibodies specific to the antigens of step j, or sites or regions of step k, or other therapeutic agents specific to the of step j, or sites or regions of step k, or to related antigens. In some embodiments, a method of treating a subject who has cancer is disclosed, the method comprising administering to said subject a therapeutically effective amount of a pharmaceutical composition comprising the medication produced using the of step j, or sites or regions identified in step k, as described above.

In some embodiments, a method of treating a subject who has cancer is disclosed, the method comprising administering to said subject a therapeutically effective amount of the isolated antibodies of step i, wherein the antibodies are either unmodified or modified.

In some embodiments, the structure or sequence, or both, of at least one of the antigens' antibody/antigen attachment sites of the antibodies/antigens complexes of step i is determined, and the antigen's antibody/antigen attachment site is used as a treatment target for non-antibody based cancer treatments. In some embodiments, at least one of the antigen's antibody/antigen attachment sites is used as a vaccine administered to an animal for creation of antibodies specific to the antigen's antibody/antigen attachment site. In some embodiments, a method of treating a subject who has cancer is disclosed, the method comprising administering to said subject a therapeutically effective amount of the said antibodies specific to the antigen's antibody/antigen attachment site, wherein the antibodies are either unmodified or modified.

In some embodiments, a method of detecting B cells which generate antibodies against the cancer cells in the subject who has cancer is disclosed, the method comprising the steps of:
a. determining the amino acid sequence of the at least one of the antigen's antibody/antigen attachment sites and synthesizing and labeling a peptide comprising at least part of the said amino acids sequence, or labeling the at least one of the antigen's antibody/antigen attachment sites; and,
b. incubating the labeled peptide or labeled at least one of the antibody/antigen attachment sites of step b with the serum obtained from the subject who has cancer, and allowing time for specific binding between the labeled peptides and B cells.

In some embodiments of the method of detecting B cells which generate antibodies against the cancer cells, the type of label of the peptide of step a is selected from a group comprising radioactive isotopes, radiolabeled amino acid, and/or fluorescent amino acids. In some embodiments, the labeled B cells are isolated, cell cultured, and induced to produce the cancer-specific antibodies. In some embodiments, a method of treating a subject who has cancer is disclosed, the method comprising administering to said subject a therapeutically effective amount of the isolated B cells. In some embodiments, a method of treating a subject who has cancer is disclosed, the method comprising administering to the said subject who has cancer a therapeutically effective amount of the antibodies, and/or their derivatives, produced by the isolated B cells.

### DETAILED DESCRIPTION

The presently disclosed subject matter now will be described more fully hereinafter. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims.

The present disclosure provides a method for utilizing a checkpoint inhibition in a subject who has cancer for generation, isolation, and identification of cancer-specific antibodies. The subject who has cancer may be, for example, a cancer patient. In some embodiments, the checkpoint inhibition is, or is part of, a treatment administered, for example, by a physician, to a cancer patient. In some other embodiments, the checkpoint inhibition is performed exclusively, or mainly, for the purpose of inducing the generation of cancer-specific antibodies in a cancer patient, and isolating the antibodies generated.

Treatment of cancer with immune checkpoint inhibitors may be less toxic and easier to tolerate than most chemotherapy drugs, and some immune checkpoint inhibitors are already in use with a wide range of cancer types. For example, a PD-1 inhibitor, Nivolumab, has been approved to treat melanoma, lung cancer, kidney cancer, bladder cancer, head and neck cancer, and Hodgkin's lymphoma. Another drug, ipilimumab-a CTLA-4 inhibitor, was approved for treatment of melanoma. Some of other types of cancers are also treated with immune checkpoint inhibitors, including breast cancer, cervical cancer, colon cancer, and liver cancer. However, even immune checkpoint inhibitors may cause side effects, especially when administered as a long term, and/or continuous, treatment. The presently disclosed disclosure provides a method of utilizing a treatment, or an acute treatment, with immune checkpoint inhibitor/s for isolating and characterizing cancer-specific antibodies generated in a cancer patient as a result of the checkpoint inhibition. The cancer-specific antibodies isolated and characterized, and/or their antigens' binding sites, may be used for creating cancer-specific medication/s aimed at killing, or inactivating, cancer cells while causing minimal harm to normal cells.

In some embodiments, the present disclosure provides a method for identifying cancer-specific antibodies utilizing an immune checkpoint inhibition treatment in a cancer patient. Generally, the presently disclosed method comprises the steps of:

a. Obtaining cancer cells and/or their components, and normal cells and/or their components from a cancer patient. Obtaining cells and or/their components from the cancer patient can be done using any suitable method known in the art, such as without limitation, biopsy. The cells and/or their components are then kept for future use. Keeping the cells and/or their components for future use may be done using methods traditional in the art, such as freezing them in a cell culture medium (such as a buffer). In some embodiments, prior to freezing the cells they are proliferated in cell culture dishes. In some other embodiments, the cells are kept for future use in cell culture dishes by continues proliferation and splitting using methods traditional in the art.
In a preferred embodiment, the cancer cells and/or their components are of the same tissue type as the normal cells and/or their components. In other embodiments, the cancer cells and/or their components are of a tissue type similar, or related, to the tissue type of the normal cells and/or their components. In yet other embodiments, the cancer cells and/or their components are of a tissue type distinct from the tissue type of the normal cells and/or their components.
b. Administering the immune checkpoint inhibition treatment to the cancer patient. In some embodiments, prescription of the immune checkpoint inhibition treatment and administration thereof is performed by the cancer patient physician/s. Immune checkpoint inhibitors are usually administered intravenously. The treatment period usually lasts 30 to 60 minutes. The number of sessions may vary depending on the immune checkpoint inhibitor/s drug being administered. In some cases, a longer-term treatment may bring about more toxic side effects then a shorter, acute, treatment. In some embodiments, the presently disclosed method results in the creation of cancer-patient-specific, and cancer-specific, medication/s which may be administered to the cancer patient whether the immune checkpoint inhibition treatment is chronic or acute. In some other preferred embodiments, the presently disclosed method provides cancer-patient-specific, and cancer-specific, medication/s which may be administered to the cancer patient, wherein the immune checkpoint inhibition treatment is acute, i.e. when the cancer patient receives the presently disclosed cancer-patient-specific, and cancer-specific, medications he/she no longer receives the immune checkpoint inhibition treatment. Thereby limiting toxicity from a long exposure to the immune checkpoint inhibition treatment to the minimum. In any of the embodiments of the present disclosure the “cancer patient” may be the cancer patient from whom the cancer cells and/or their components, and normal cells and/or their components were obtained, or any other cancer patient, or any subject in need.
c. Following the commencement of the immune checkpoint inhibition treatment, wherein the treatment comprises at least one administration of immune checkpoint inhibitor/s drugs, time is allowed for the cancer patient's immune system to react against the cancer cells. In some embodiments, the time following treatment is, or is about, 2-8 weeks. In some other embodiments, the time following treatment is, or is about, 4, 5, or 6 weeks.
d. Obtaining a blood sample from the cancer patient, wherein the sample may comprise antibodies which may have been generated against the patient's cancer cells. A serum comprising the antibodies present in the blood sample may be prepared from the blood sample using methods traditional in the art. For example, the blood may be centrifuged to separate the serum, the serum then may be filtered to remove clots and particulate matter. In some embodiments, the serum is then used for the following steps. In some other embodiments, the serum is then used to prepare a solution, wherein the solution comprises the antibodies present in the serum, and is used for the following steps. For example, the serum may be treated with supersaturated ammonium sulfate (45% solution at room temperature) to precipitate the antibodies and other material. The resulting solution is centrifuged at 5000 rpm for five minutes, after which the supernatant fluid is removed. The precipitated proteins (including antibodies) are resuspended in phosphate-buffered saline (PBS buffer).
In some embodiments, the serum or solution, will contain various types of antibodies including:

1. Antibodies that react (cross react) with both cancer and normal cells.

2. Antibodies that react only with cancer cells.

3. Antibodies that are specific for the old agents (bacteria, viruses, etc.) that the cancer patient was exposed to.

e. Optionally, discontinuing the immune checkpoint inhibition treatment of the cancer patient. In some embodiments, wherein the immune checkpoint inhibition treatment was performed for the purpose of generating antibodies, the immune checkpoint inhibition treatment may be terminated. In some other embodiments, the decision of whether to continue or discontinue the immune checkpoint inhibition treatment is made by the cancer patient physician/s.
f. Removing from the serum, or solution, of step d antibodies that bind to the normal cells and/or components. In a preferred embodiment, this done by incubating the serum, or solution, with a cell culture prepared from the normal cells and/or their components of step a, allowing sufficient time for antibodies/antigens binding and generation of antibodies/antigen complexes. Antibodies that have an affinity for normal cells or their components will bind to them and therefore be removed from the solution or serum. The solution or serum may then comprise antibodies that have an affinity for the cancer cells and/or their components of the cancer patient of step a, or antibodies that were created by the cancer patient in response to old agents. The supernatant (i.e. serum or solution) is then removed from the tissue culture and kept, to be used in step g.
g. The antibodies that bind to the cancer cells and/or their components of step a are selected. In some embodiments, the selection is performed by incubating the supernatant of step f with a culture of the cancer cells and/or their components of step a, allowing sufficient time for antibodies/antigens binding and generation of antibodies/antigen complexes. The supernatant is removed and the culture comprising the antibodies/antigen complexes is kept for further analysis, for example as described in the following steps.

The incubations of step f and g, and generally incubations described in this disclosure, are performed using any suitable protocol known in the art for incubations aimed to enable antibody/antigen binding. In some embodiments, the incubation is performed in room temperature, or 37° C. In some embodiments, the incubation length is in the range of 10 minutes to 72 hours. The incubation may be for 1-12 hours at room temperature, for example 1, 2, 3, 4, 5, 6, 7, 8, 9, 10, 11, or 12 hours. More specifically, in some embodiments, the incubation is done “overnight”, within the range of 12-24 hours. In some embodiments, the incubation is done overnight at 4° C.

h. The antibody/antigen complexes of step g are extracted. In some embodiments, this is done by fractionating the cell culture of step g following the incubation and removal of the supernatant. In some embodiments, prior to fractionating, a cell lysate is prepared of the cell culture of step g (following the incubation and removal of the supernatant). The conditions used for prepare the lysate should be gentle enough to retain the antibody binding sites but strong enough to quantitatively solubilize the antigen of interest. For example, the lysis buffer may contain salt concentrations between 0-1M, nonionic detergent concentrations between 0.1-2%, divalent cation concentrations between 0-10 mM, EDTA concentrations between 0-5 mM, and pHs between 6-9. In addition, an antiprotease cocktail may be included.

In some embodiments, the cancer cells and/or their components and normal cells and/or their components obtained from the cancer patient in step a are kept for future use in the form of a cell lysate, which may be prepared using any method known in the art and/or described herein. Subsequently, the incubation of step f comprises incubating serum or solution with a lysate of normal cells and/or their components, and the incubation of step g comprises incubating the supernatant of step f with a lysate of cancer cells and/or their components. Therefore, in some embodiments, preparation of a lysate in the present step is not necessary.

The antibody/antigen complexes may be separated from the rest of the lysate (described above), or a solution prepared of the lysate, using protocols standard in the art. In some embodiments, suitable protocols include, without limitation, affinity chromatography, or co-immunoprecipitation. Co-IP generally involves capturing the immune complex, or precipitating it, for example, on a beaded support to which an antibody-binding protein is immobilized (such as Protein A or G), and any proteins not precipitated on the beads are washed away. Otherwise, the antibody/antigen are simply precipitated without the use of beads. Optionally then, the antigen (and antibody, if it is not covalently attached to the beads and/or when using denaturing buffers) may be eluted from the support and analyzed by sodium dodecyl sulfate-polyacrylamide gel electrophoresis (SDS-PAGE), often followed by western blot detection or mass spectrometry to verify the identity of the antigen.

i. The antibodies and the antigens of the antibody/antigen complexes of step h are separated using protocols standard in the art. In some embodiments, methods used for separation of the antibodies and antigens include, without limitation, western blot, or chromatography. In some embodiments, the separated antibodies of this step may be administrated to the cancer patient of step a to fight the cancer cells his/her body. Since the antibodies are made by the cancer patient of step a, they should be able to be used therapeutically without eliciting an adverse immune response. In some embodiment, the antibodies administered to the said cancer patient may be modified (e.g. conjugated to a toxic drug).
j. The antigens of step i are analyzed using standard methods for antigen identification. Further to the separation described above, the antibodies may be further purified, e.g., using filtration, centrifugation and various chromatographic methods, such as HPLC or affinity chromatography, all of which purification techniques are well known to those of skill in the art. These purification techniques each involve fractionation to separate the desired antibody from other components of a mixture. Analytical methods particularly suited to the preparation of antibodies include, for example, protein A-Sepharose and/or protein G-Sepharose chromatography.
k. Sites, or regions, on the antigens of step j which are specific to the cancer cells or their components obtained from the cancer patient of step a are identified and analyzed. This may be done using any suitable method for antigen identification traditional in the art, such as, without limitation, western blot, ELISA, gel shift assays, reporter assays, immunospectroscopy, sequencing (such as, protein or nucleic acids sequencing), synthesis (such as, protein or nucleic acids synthesis), mass spectrometry, and combinations thereof.

The antigens identified in step k may be used to produce medication/s. in some embodiments, the medication/s are substantially specific to cancer cells (i.e. specifically kills, or inhibit, cancer cells), and in some instances it is specific, or also specific, to the cancer cells of the cancer patient of step a. In some embodiments, the medication/s may be antibodies specific to the cancer-specific antigens. In other embodiments, the medication/s may be other therapeutic agents specific to the cancer-specific antigens, or to related antigens.

For example, treatment/medication-creation approaches may include, without limitation:
The cancer-specific antibodies, or otherwise monoclonal antibodies derived from them, could be used in a method of treatment to directly target and kill, or inhibit, the cancer cells.
Custom designed cytotoxic drugs may be developed to target the cancer-specific antigens, or specific regions of the cancer-specific antigens, and used to kill, or inhibit, the cancer cells that comprise them.
The cancer-specific antigens, or specific regions of the cancer-specific antigens, may be used to create a vaccine that may be administered to a cancer patient and stimulate the body's own immune system to kill the cancer cells. It is also contemplated herein that the vaccine, or an embodiment of this vaccine, may be given to healthy subjects, or subjects who are at risk of having cancer, for the purpose of prevention a development of cancer. As an example, a specific region of a cancer-specific antigen may be the antigen's antibody/antigen attachment site.
Some combination of these therapeutic approaches, or others.

In some embodiments, the isolated antibodies of step i, possibly after being pharmaceutically formulated, may be injected directly to the cancer patient of step a. In some embodiments, the antibodies are either unmodified or modified. Modification may include for example, conjugation of the antibodies to moieties such as drugs. For example, oncology drugs. In some embodiments, the structure or sequence, or both, of at least one of the antibody/antigen attachment sites of the antibodies/antigens complexes of step i is determined, and the antibody/antigen attachment site is used as a treatment target for non-antibody based cancer treatments/oncology drugs.

In some embodiments, the cancer-specific antigen's antibody/antigen attachment site is formulated as a vaccine which may be administered to a non-human animal for creation of antibodies specific to the antigen's antibody/antigen attachment site. Subsequently, the animal may generate antibodies specific to the antigen's antibody/antigen attachment site. These antibodies may be administered to an subject in need, such as a cancer patient. The antibodies may be either modified or unmodified. In any of the embodiments of the present disclosure, modification includes conjugation of the antibodies to any desired moieties, such as drugs/toxins. The animal may be any animal traditionally used for creation of antibodies, and the vaccination of the animal may be done using any method traditional in the field.

In some embodiments, the B cells which generate antibodies against the cancer cells in the cancer patient may be detected and isolated. Detection of the B cells may comprise the steps of:

1. Determining the amino acid sequence of a cancer-specific antigen's antibody/antigen attachment site and synthesizing and labeling a peptide comprising at least part of the said amino acids sequence. Alternatively, labeling the at least one of the identified antibody/antigen attachment sites.

2. incubating the labeled peptide of step 2 with the serum obtained from the cancer patient, or with the solution prepared from the serum, and allowing enough time for specific binding between the labeled peptides and B cells.

The labeling of the peptide of step 1 may comprise any type of suitable label. For example, the label may be radioactive isotopes, radiolabeled amino acid, fluorescent amino acids, or combinations thereof. The peptide-bound, and therefore labeled, B cells may be isolated, proliferated using cell culture techniques, and induced to produce antibodies, for example, cancer-specific antibodies. Either the B cells themselves, or the cancer-specific antibodies produced by them, may be pharmaceutically formulated and administered in therapeutically effective amount/s to a subject in need, for example a/the cancer patient. Importantly, when the B cells themselves, or the cancer-specific antibodies produced by them, are introduced into the cancer patient of step a, no adverse immune response should be elicited. In any of the embodiments of the present disclosure, modifications or derivations of the cancer-specific antibodies may be created and used as medication/s.

In the present description, references to human cancer are also applicable to cancer in any other species. By “normal cell” is generally meant non-cancerous cells.

In some embodiments, for the preparation of a vaccine an immunizing solution which is formulized for injection into an animal (including human) is created. In some embodiments, the said solution comprises at least one adjuvant. In some embodiments, the immunizing solution is PBS (phosphate-buffered saline) based.

Injections, for example for the purpose of vaccination, are administered according to protocols traditionally used in the art. With regard to non-human animals, the injections may be Subcutaneous (SC), Intramuscular (IM), Intraperitoneal (IP), or Intradermal (ID). Injections for routine antibody production may be administered subcutaneously in two to four sites per animal, generally on the back, away from the spine.

By “treating” is meant ameliorating at least one symptom of a condition or disease in a subject having the condition or disease (e.g., a subject diagnosed with cancer), as compared with an equivalent untreated control. Such reduction in the symptom (e.g., a reduction in tumor size or metastasis) is at least 5%, 10%, 20%, 40%, 50%, 60%, 80%, 90%, 95%, or 100%, as measured by any standard technique.

While certain specific details of the construction and material selection have been disclosed herein, these will suggest many variations to those skilled in the art. It is not intended to limit this invention to the precise details disclosed herein. It will be apparent to those skilled in the art that many modifications and substitutions can be made to the preferred embodiments just described without departing from the spirit and scope of the invention as defined in the appended claims.

By “effective amount” or “therapeutically effective amount” is meant an amount of a molecule, compound or antibody required to treat, treat prophylactically, or reduce disease or disorder in a clinically relevant manner. For example, an effective amount of an active compound used according to the present invention varies depending upon the manner of administration, the age, body weight, and general health of the subject. Ultimately, the prescribers will decide the appropriate amount and dosage regimen.

By “subject”, or a “subject in need” is meant a human or non-human animal (e.g., a mammal).

The terms “antibody” as used herein, refer broadly to any immunological binding agent, including polyclonal and monoclonal antibodies. Depending on the type of constant domain in the heavy chains, antibodies are assigned to one of five major classes: IgA, IgD, IgE, IgG, and IgM. Several of these are further divided into subclasses or isotypes, such as IgG1, IgG2, IgG3, IgG4, and the like. The heavy-chain constant domains that correspond to the difference classes of immunoglobulins are termed α, δ, ε, γ and μ, respectively. The subunit structures and three-dimensional configurations of different classes of immunoglobulins are well known.

Generally, where antibodies rather than antigen binding regions are used in the invention, IgG and/or IgM are preferred because they are the most common antibodies in the physiological situation and because they are most easily made in a laboratory setting. The “light chains” of mammalian antibodies are assigned to one of two clearly distinct types: kappa (κ) and lambda (λ), based on the amino acid sequences of their constant domains. There is essentially no preference to the use of κ or λ light chains in the antibodies of the present invention.

For the purposes of the present disclosure, cell culture medium is a media suitable for growth of animal cells, such as mammalian cells, in in vitro cell culture. Cell culture media formulations are well known in the art. Typically, cell culture media are comprised of buffers, salts, carbohydrates, amino acids, vitamins and trace essential elements. The cell culture medium may or may not contain serum, peptone, and/or proteins. Various tissue culture media, including serum-free and defined culture media, are commercially available, for example, any one or a combination of the following cell culture media can be used: RPMI-1640 Medium, RPMI-1641 Medium, Dulbecco's Modified Eagle's Medium (DMEM), Minimum Essential Medium Eagle, F-12K Medium, Ham's F12 Medium, Iscove's Modified Dulbecco's Medium, McCoy's 5A Medium, Leibovitz's L-15 Medium, and serum-free media such as EX-CELL™ 300 Series (JRH Biosciences, Lenexa, Kans.), among others. Cell culture media may be supplemented with additional or increased concentrations of components such as amino acids, salts, sugars, vitamins, hormones, growth factors, buffers, antibiotics, lipids, trace elements and the like, depending on the requirements of the cells to be cultured and/or the desired cell culture parameters.

Cell culture media may be serum-free, protein-free, and/or peptone-free. “Serum-free” applies to a cell culture medium that does not contain animal sera, such as fetal bovine serum. “Protein-free” applies to cell culture media free from exogenously added protein, such as transferrin, protein growth factors IGF-1, or insulin. Protein-free media may or may not contain peptones. “Peptone-free” applies to cell culture media which contains no exogenous protein hydrolysates such as animal and/or plant protein hydrolysates. Eliminating serum and/or hydrolysates from cell culture media has the advantage of reducing lot to lot variability and enhancing processing Steps, such as filtration. However, when serum and/or peptone are removed from the cell culture media, cell growth, viability and/or protein expression may be diminished or less than optimal. As such, serum-free and/or peptone-free cell culture medium may be highly enriched for amino acids, trace elements and the like. See, for example, U.S. Pat. Nos. 5,122,469 and 5,633,162.

Although there are many media formulations, there is a need to develop defined media formulations that perform as well or preferably better than those containing animal sera and/or peptones.

Defined cell culture media formulations are complex, containing amino acids, inorganic salts, carbohydrates, lipids, vitamins, buffers and trace essential elements. Identifying the components that are necessary and beneficial to maintain a cell culture with desired characteristics is an ongoing task. Defined basal media formulations which are supplemented or enriched to meet the needs of a particular host cell or to meet desired performance parameters is one approach to developing defined media. Identifying those components and optimum concentrations that lead to improved cell growth, viability and protein production is an ongoing task.

By cell culture or “culture” is meant the growth and propagation of cells outside of a multicellular organism or tissue. Suitable culture conditions for mammalian cells are known in the art. See e.g. Animal cell culture: A Practical Approach, D. Rickwood, ed., Oxford University Press, New York (1992). Mammalian cells may be cultured in suspension or while attached to a solid substrate. Fluidized bed bioreactors, hollow fiber bioreactors, roller bottles, shake flasks, or stirred tank bioreactors, with or without microcarriers, and operated in a batch, fed batch, continuous, semi-continuous, or perfusion mode are available for mammalian cell culture.

Mammalian cells, such as CHO cells, may be cultured in small scale cultures, such as for example, in 100 ml containers having about 30 ml of media, 250 ml containers having about 80 to about 90 ml of media, 250 ml containers having about 150 to about 200 ml of media.

Alternatively, the cultures can be large scale such as for example 1000 ml containers having about 300 to about 1000 ml of media, 3000 ml containers having about 500 ml to about 3000 ml of media, 8000 ml containers having about 2000 ml to about 8000 ml of media, and 15000 ml containers having about 4000 ml to about 15000 ml of media. Large scale cell cultures, such as for clinical manufacturing of protein therapeutics, are typically maintained for days, or even weeks, while the cells produce the desired protein(s).

During this time the culture can be supplemented with a concentrated feed medium containing components, such as nutrients and amino acids, which are consumed during the course of the production phase of the cell culture. Concentrated feed medium may be based on just about any cell culture media formulation. Such a concentrated feed medium can contain most of the components of the cell culture medium at, for example, about 5×, 6×, 7×, 8×, 9×, 10×, 12×, 14×, 16×, 20×, 30×, 50×, 100×, 200×, 400×, 600×, 800×, or even about 1000× of their normal amount. Concentrated feed media are often used in fed batch culture processes.

The best temperature for culturing cells is related to the body temperature of the animal the cells were derived from. Typical temperatures for cell cultures derived from animals are: mammals 36° C. to 37° C., birds 38.5° C., cold blooded vertebrates such as fish, amphibians and reptiles between 15° C. and 26° C.

In some embodiments of any of the methods described herein, a polypeptide may be used in any of the methods of analyzing the antigens of the present disclosure. In some embodiments, the polypeptide is an antibody, such as T cell-dependent bispecific (TDB) antibody.

Molecular targets for antibodies include CD proteins and their ligands, such as, but not limited to: (i) CD3, CD4, CDS, CD19, CD11 a, CD20, CD22, CD34, CD40, CD79a (CD79a), and CD79|3 (CD79b); (ii) members of the ErbB receptor family such as the EGF receptor, HER2, HERS or HER4 receptor; (iii) cell adhesion molecules such as LFA-1, Macl, pl50,95, VLA-4, ICAM-1, VCAM and αv./β3 integrin, including either alpha or beta subunits thereof (e.g. , anti-CD11a, anti-CD18 or anti-CD1 ib antibodies): (iv) growth factors such as VEGF; IgE; blood group antigens; flk2/flt3 receptor; obesity (OB) receptor; mpl receptor; CTLA-4; protein C, BR3, c-met, tissue factor, (37 etc; (v) cell surface and transmembrane tumor-associated antigens (TAA), such as those described in U.S. Pat. No. 7,521,541 , and (vi) other targets such as FcRH5, LyPD1, TenB2 and STEAP. In some embodiments, the antibody is an anti-CD20/anti-CD3 antibody. Other exemplar' antibodies include those selected from, and without limitation, anti-estrogen receptor antibody, anti-progesterone receptor antibody, anti-p53 antibody, anti-HER-2/neu antibody, anti-EGFR antibody, anti-cathepsm D antibody, anti-Bcl-2 antibody, anti-E-cadherin antibody, anti-CA, 25 antibody, anti-CA15-3 antibody, anti-CA19-9 antibody, anti-c-erbB-2 antibody, anti-P-glycoprotein antibody, anti-CEA antibody, anti-retinoblastoma protein antibody, anti-ras oncoprotein antibody, anti-Lewis X antibody, anti-Ki-67 antibody, anti- PCNA antibody, anti-CD3 antibody, anti-CD4 antibody, anti-CD5 antibody, anti-CD7 antibody, anti-CD8 antibody, anti-CD9/p24 antibody, anti-CD10 antibody, anti-CD!1a antibody, anti-CD11c antibody, anti-CD13 antibody, anti-CD14 antibody, anti-CD15 antibody, anti-CD19 antibody, anti-CD20 antibody, anti-CD22 antibody, anti-CD23 antibody, anti-CD30 antibody, anti-CD31 antibody, anti-CD33 antibody, anti-CD34 antibody, anti-CD35 antibody, anti-CD38 antibody, a ti-CD4i antibody, anti-LCA/CD45 antibody, anti-CD45RO antibody, anti- CD45RA antibody, anti-CD39 antibody, anti-CD100 antibody, anti˜CD95/Fas antibody, anti-CD99 antibody, anti-CD106 antibody, anti-ubiquitin antibody, anti-CD71 antibody, anti-c-myc antibody, anti-cytokeratins antibody, anti-vimentin antibody, anti-HPV proteins antibody, anti-kappa light chains antibody, anti-lambda light chains antibody, anti-melanosomes antibody, anti-prostate specific antigen antibody, anti-S-100 antibody, anti-tau antigen antibody, anti-fibrin antibody, anti-keratins antibody, anti-TebB2 antibody, anti-STEAP antibody, and anti-Tn-antigen antibody.

By “cancer” is meant any condition diseases involving abnormal cell growth with the potential to invade or spread to other parts of the body. Cancer according to the present disclosure is, but not limited to Acute Lymphoblastic Leukemia (ALL), Acute Myeloid Leukemia (AML), Adrenocortical Carcinoma, Childhood Adrenocortical Carcinoma, AIDS-Related Cancers, Kaposi Sarcoma (Soft Tissue Sarcoma), AIDS-Related Lymphoma (Lymphoma), Primary CNS Lymphoma (Lymphoma), Anal Cancer, Appendix Cancer, Astrocytomas, Childhood Brain Cancer, Atypical Teratoid/Rhabdoid Tumor, Central Nervous System (Brain Cancer), Basal Cell Carcinoma of the Skin, Bile Duct Cancer, Bladder Cancer, Childhood Bladder Cancer, Bone Cancer (includes Ewing Sarcoma and Osteosarcoma and Malignant Fibrous Histiocytoma), Brain Tumors, Breast Cancer, Childhood Breast Cancer, Bronchial Tumors, Burkitt Lymphoma, Non-Hodgkin Lymphoma, Carcinoid Tumor (Gastrointestinal), Childhood Carcinoid Tumors, Carcinoma of Unknown Primary, Childhood Carcinoma of Unknown Primary, Cardiac (Heart) Tumors, Central Nervous System cancer, Atypical Teratoid/Rhabdoid Tumor, Embryonal Tumors, Germ Cell Tumor, Primary CNS Lymphoma, Cervical Cancer, Childhood Cervical Cancer, Cholangiocarcinoma, Bile Duct Cancer, Chordoma, Chronic Lymphocytic Leukemia (CLL), Chronic Myelogenous Leukemia (CML), Chronic Myeloproliferative Neoplasms, Colorectal Cancer, Childhood Colorectal Cancer, Craniopharyngioma, Cutaneous T-Cell Lymphoma—(Mycosis Fungoides and Sézary Syndrome), Ductal Carcinoma In Situ (DCIS), Embryonal Tumors, Endometrial Cancer (Uterine Cancer), Ependymoma, Esophageal Cancer, Childhood Esophageal Cancer, Esthesioneuroblastoma, Ewing Sarcoma (Bone Cancer), Extracranial Germ Cell Tumor, Extragonadal Germ Cell Tumor, Eye Cancer, Childhood Intraocular Melanoma, Intraocular Melanoma, Retinoblastoma, Fallopian Tube Cancer, Fibrous Histiocytoma of Bone-Malignant, Osteosarcoma, Gallbladder Cancer, Gastric (Stomach) Cancer, Childhood Gastric (Stomach) Cancer, Gastrointestinal Carcinoid Tumor, Gastrointestinal Stromal Tumors (GIST) (Soft Tissue Sarcoma), Childhood Gastrointestinal Stromal Tumors, Childhood Central Nervous System Germ Cell Tumors, Childhood Extracranial Germ Cell Tumors, Extragonadal Germ Cell Tumors, Ovarian Germ Cell Tumors, Testicular Cancer, Gestational Trophoblastic Disease, Hairy Cell Leukemia, Head and Neck Cancer, Childhood Head and Neck Cancers, Heart Tumors, Hepatocellular (Liver) Cancer, Histiocytosis, Langerhans Cell, Hodgkin Lymphoma, Hypopharyngeal Cancer (Head and Neck Cancer), Intraocular Melanoma, Childhood Intraocular Melanoma, Islet Cell Tumors, Pancreatic Neuroendocrine Tumors, Kaposi Sarcoma (Soft Tissue Sarcoma), Kidney (Renal Cell) Cancer, Langerhans Cell Histiocytosis, Laryngeal Cancer (Head and Neck Cancer), Childhood Laryngeal Cancer and Papillomatosis, Leukemia, Lip and Oral Cavity Cancer (Head and Neck Cancer), Liver Cancer, Lung Cancer (Non-Small Cell and Small Cell), Childhood Lung Cancer, Lymphoma, Male Breast Cancer, Malignant Fibrous Histiocytoma of Bone and Osteosarcoma, Melanoma, Childhood Melanoma, Melanoma, Intraocular (Eye), Childhood Intraocular Melanoma, Merkel Cell Carcinoma (Skin Cancer), Mesothelioma, Malignant Childhood Mesothelioma, Metastatic Cancer, Metastatic Squamous Neck Cancer with Occult Primary (Head and Neck Cancer), Midline Tract Carcinoma Involving NUT Gene, Mouth Cancer (Head and Neck Cancer), Multiple Endocrine Neoplasia Syndromes, Multiple Myeloma/Plasma Cell Neoplasms, Mycosis Fungoides (Lymphoma), Myelodysplastic Syndromes, Myelodysplastic/Myeloproliferative Neoplasms, Myelogenous Leukemia, Chronic (CML), Myeloid Leukemia, Acute (AML), Chronic Myeloproliferative Neoplasms, Nasal Cavity and Paranasal Sinus Cancer (Head and Neck Cancer), Nasopharyngeal Cancer (Head and Neck Cancer), Childhood Nasopharyngeal Cancer, Neuroblastoma, Non-Hodgkin Lymphoma, NonSmall Cell Lung Cancer, Oral Cancer, Lip and Oral Cavity Cancer and Oropharyngeal Cancer (Head and Neck Cancer), Childhood Oral Cavity Cancer, Osteosarcoma and Malignant Fibrous Histiocytoma of Bone, Ovarian Cancer, Childhood Ovarian Cancer, Pancreatic Cancer, Childhood Pancreatic Cancer, Pancreatic Neuroendocrine Tumors (Islet Cell Tumors), Papillomatosis, Paraganglioma, Childhood Paraganglioma, Paranasal Sinus and Nasal Cavity Cancer (Head and Neck Cancer), Parathyroid Cancer, Penile Cancer, Pharyngeal Cancer (Head and Neck Cancer), Pheochromocytoma, Childhood Pheochromocytoma, Pituitary Tumor, Plasma Cell Neoplasm/Multiple Myeloma, Pleuropulmonary Blastoma, Pregnancy and Breast Cancer, Primary Central Nervous System (CNS) Lymphoma, Primary Peritoneal Cancer, Prostate Cancer, Rectal Cancer, Recurrent Cancer, Renal Cell (Kidney) Cancer, Retinoblastoma, Childhood Rhabdomyosarcoma (Soft Tissue Sarcoma), Salivary Gland Cancer (Head and Neck Cancer), Childhood Salivary Gland Tumors, Sarcoma, Childhood Rhabdomyosarcoma (Soft Tissue Sarcoma), Childhood Vascular Tumors (Soft Tissue Sarcoma), Ewing Sarcoma (Bone Cancer), Kaposi Sarcoma (Soft Tissue Sarcoma), Osteosarcoma (Bone Cancer), Uterine Sarcoma, Sézary Syndrome (Lymphoma), Skin Cancer, Childhood Skin Cancer, Small Cell Lung Cancer, Small Intestine Cancer, Soft Tissue Sarcoma, Squamous Cell Carcinoma of the Skin, Squamous Neck Cancer with Occult Primary, Metastatic (Head and Neck Cancer), Stomach (Gastric) Cancer, Childhood Stomach (Gastric) Cancer, T-Cell Lymphoma, Cutaneous (Mycosis Fungoides and Sezary Syndrome), Testicular Cancer, Childhood Testicular Cancer, Throat Cancer (Head and Neck Cancer), Nasopharyngeal Cancer, Oropharyngeal Cancer, Hypopharyngeal Cancer, Thymoma and Thymic Carcinoma, Thyroid Cancer, Childhood Thyroid Tumors , Transitional Cell Cancer of the Renal Pelvis and Ureter (Kidney (Renal Cell) Cancer), Childhood Cancer of Unknown Primary, Ureter and Renal Pelvis, Transitional Cell Cancer, Kidney Renal Cell Cancer, Urethral Cancer, Endometrial Uterine Cancer, Uterine Sarcoma, Vaginal Cancer, Childhood Vaginal Cancer, Vascular Tumors (Soft Tissue Sarcoma), Vulvar Cancer, and Wilms Tumor and Other Childhood Kidney Tumors.

The therapeutic agents or compositions of the disclosure are given on a per diem basis but should not be interpreted as necessarily being administered on a once daily frequency.

Indeed, the therapeutic agents, compositions, compound, salt or prodrug thereof, can be administered at any suitable frequency, for example as determined conventionally by a physician taking into account a number of factors, but typically about four times a day, three times a day, twice a day, once a day, every second day, twice a week, once a week, twice a month or once a month. In some situations, a single dose may be administered, but more typically administration is according to a regimen involving repeated dosage over a treatment period. In such a regimen the daily dose and/or frequency of administration can, if desired, be varied over the course of the treatment period, for example introducing the subject to the compound, composition, salt or prodrug thereof at a relatively low dose and then increasing the dose in one or more Steps until a full dose is reached. The treatment period is generally as long as is needed to achieve a desired outcome.

It will generally be found preferable to administer the (Active Pharmaceutical Ingredient-therapeutic agent, compounds and compositions of the present disclosure) API in a pharmaceutical composition that comprises the API and at least one pharmaceutically acceptable excipient. The excipient(s) collectively provide a vehicle or carrier for the API. Pharmaceutical compositions adapted for all possible routes of administration are well known in the art and can be prepared according to principles and procedures set forth in standard texts and handbooks such as those individually cited below:

USIP, ed. (2005) Remington: The Science and Practice of Pharmacy, 21st ed., Lippincott, Williams & Wilkins.

Allen et al. (2004) Ansel's Pharmaceutical Dosage Forms and Drug Delivery Systems, 8th ed., Lippincott, Williams & Wilkins.

Suitable excipients are described, for example, in Kibbe, ed. (2000) Handbook of Pharmaceutical Excipients, 3rd ed., American Pharmaceutical Association.

Examples of formulations that can be used as vehicles for delivery of the API in practice of the present disclosure include, without limitation, solutions, suspensions, powders, granules, tablets, capsules, pills, lozenges, chews, creams, ointments, gels, liposomal preparations, nanoparticulate preparations, injectable preparations, enemas, suppositories, inhalable powders, sprayable liquids, aerosols, patches, depots and implants.

For oral delivery, the API can be formulated in liquid or solid form, for example as a solid unit dosage form such as a tablet or capsule. Such a dosage form typically comprises as excipients one or more pharmaceutically acceptable diluents, binding agents, disintegrants, wetting agents and/or antifrictional agents (lubricants, anti-adherents and/or glidants). Many excipients have two or more functions in a pharmaceutical composition. Characterization herein of a particular excipient as having a certain function, e.g., diluent, binding agent, disintegrant, etc., should not be read as limiting to that function.

Concerning all methods, the terms “a” and “an” are used to mean “at least one”, “at least a first”, “one or more” or “a plurality” of steps in the recited methods, except where specifically stated.

It is expressly contemplated that the methods described herein are not limited for the creation of antibodies specific to cancer antigens, but may be used for the creation and selection of target-specific antibodies against any desired antigen, such as without limitation, a viral antigen.

### EXAMPLE 1

### Detection of Antigens

For the detection of protein biomarkers various protein assays are available including, for example, antibody-based methods as well as mass spectroscopy and other similar means known in the art. In the case of antibody-based methods, for example, the sample may be contacted with an antibody specific for said antigen under conditions sufficient for an antibody-antigen complex to form, and then detecting said complex. Detection of the presence of the protein antigen may be accomplished in a number of ways, such as by Western blotting (with or without immunoprecipitation), 2-dimensional SDS-PAGE, immunoprecipitation, fluorescence activated cell sorting (FACS), flow cytometry, and ELISA procedures for assaying a wide variety of tissues and samples, including plasma or serum. A wide range of immunoassay techniques using such an assay format are available, see, e.g., U.S. Pat. Nos. 4,016,043, 4,424,279, and 4,018,653. These include both single-site and two-site or “sandwich” assays of the non-competitive types, as well as in the traditional competitive binding assays. These assays also include direct binding of a labeled antibody to a target antigen.

Sandwich assays are among the most useful and commonly used assays. A number of variations of the sandwich assay technique exist, and all are intended to be encompassed by the present invention. Briefly, in a typical forward assay, an unlabeled antibody is immobilized on a solid substrate, and the sample to be tested brought into contact with the bound molecule. After a suitable period of incubation, for a period of time sufficient to allow formation of an antibody-antigen complex, a second antibody specific to the antigen, labeled with a reporter molecule capable of producing a detectable signal is then added and incubated, allowing time sufficient for the formation of another complex of antibody-antigen-labeled antibody. Any unreacted material is washed away, and the presence of the antigen is determined by observation of a signal produced by the reporter molecule. The results may either be qualitative, by simple observation of the visible signal, or may be quantitated by comparing with a control sample containing known amounts of biomarker.

Variations on the forward assay include a simultaneous assay, in which both sample and labeled antibody are added simultaneously to the bound antibody. These techniques are well known to those skilled in the art, including any minor variations as will be readily apparent. In a typical forward sandwich assay, a first antibody having specificity for the biomarker is either covalently or passively bound to a solid surface. The solid surface is typically glass or a polymer, the most commonly used polymers being cellulose, polyacrylamide, nylon, polystyrene, polyvinyl chloride, or polypropylene. The solid supports may be in the form of tubes, beads, discs of microplates, or any other surface suitable for conducting an immunoassay. The binding processes are well-known in the art and generally consist of cross-linking covalently binding or physically adsorbing, the polymer-antibody complex is washed in preparation for the test sample. An aliquot of the sample to be tested is then added to the solid phase complex and incubated for a period of time sufficient (e.g., 2-40 minutes or overnight if more convenient) and under suitable conditions (e.g., from room temperature to 40° C. such as between 25° C. and 32° C. inclusive) to allow binding of any subunit present in the antibody. Following the incubation period, the antibody subunit solid phase is washed and dried and incubated with a second antibody specific for a portion of the biomarker. The second antibody is linked to a reporter molecule which is used to indicate the binding of the second antibody to the molecular marker.

An alternative method involves immobilizing the target biomarkers in the sample and then exposing the immobilized target to specific antibody which may or may not be labeled with a reporter molecule. Depending on the amount of target and the strength of the reporter molecule signal, a bound target may be detectable by direct labeling with the antibody. Alternatively, a second labeled antibody, specific to the first antibody is exposed to the target-first antibody complex to form a target-first antibody-second antibody tertiary complex. The complex is detected by the signal emitted by the reporter molecule. By “reporter molecule,” as used in the present specification, is meant a molecule which, by its chemical nature, provides an analytically identifiable signal which allows the detection of antigen-bound antibody. The most commonly used reporter molecules in this type of assay are either enzymes, fluorophores or radionuclide containing molecules (i.e., radioisotopes) and chemiluminescent molecules.

In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, beta-galactosidase, and alkaline phosphatase, amongst others. The substrates to be used with the specific enzymes are generally chosen for the production, upon hydrolysis by the corresponding enzyme, of a detectable color change. Examples of suitable enzymes include alkaline phosphatase and peroxidase. It is also possible to employ fluorogenic substrates, which yield a fluorescent product rather than the chromogenic substrates noted above. In all cases, the enzyme-labeled antibody is added to the first antibody-molecular marker complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of biomarker which was present in the sample. Alternately, fluorescent compounds, such as fluorescein and rhodamine, may be chemically coupled to antibodies without altering their binding capacity.

When activated by illumination with light of a particular wavelength, the fluorochrome-labeled antibody adsorbs the light energy, inducing a state to excitability in the molecule, followed by emission of the light at a characteristic color visually detectable with a light microscope. As in the EIA, the fluorescent labeled antibody is allowed to bind to the first antibody-molecular marker complex. After washing off the unbound reagent, the remaining tertiary complex is then exposed to the light of the appropriate wavelength, the fluorescence observed indicates the presence of the molecular marker of interest. Immunofluorescence and EIA techniques are both very well established in the art. However, other reporter molecules, such as radioisotope, chemiluminescent or bioluminescent molecules, may also be employed.

### EXAMPLE 2

### Production of Serum and Solutions Containing Antibodies

The following procedure or a variation can be used for any vertebrate. A goat is inoculated by intramuscular injection with cancer cells or their derivatives using an intramuscular injection. Blood samples are drawn after an appropriate interval, such as two weeks, for initial assessment. In the optimized procedure, the goat is injected every week for four weeks, then at six weeks the animal is then bled to obtain the reagent.

Approximately 400 cc of blood is drawn from the goat under sterile technique. The area for needle extraction is shaved and prepared with betadine. An 18-gage needle is used to draw approximately 400 cc of blood from the animal. Of note is that the animal can tolerate approximately 400 cc of blood drawn without the animal suffering any untoward effects. The animal does not have to be sacrificed. The animal can then be re-bled in approximately 10 to 14 days after it replenishes its blood volume.

The presence of potentially useful antibodies is confirmed. Once the presence of such reagents is confirmed blood is then taken from the goat at between 4-6 weeks, and centrifuged to separate the serum. 300 ml of serum is then filtered to remove large clots and particulate matter.

The serum is then treated with supersaturated ammonium sulfate (45% solution at room temperature) to precipitate antibodies and other material. The resulting solution is centrifuged at 5000 rpm for five minutes, after which the supernatant fluid is removed. The precipitated immunoglobulin is resuspended in phosphate-buffered saline (‘PBS buffer’, see Sambrook et. al. ‘Molecular cloning, A Laboratory Manual’, 1989) sufficient to re-dissolve the precipitate.

The solution is then dialyzed through a membrane with a molecular weight cut off of 10,000 Daltons. Dialysis is carried out in PBS buffer, changed every four hours over a period of 24 hours. Dialysis is carried out at 4° C.

After 24 hours of dialysis the contents of the dialysis bag are emptied into a sterile beaker. The solution is adjusted such that the mass per unit volume=10 mg per ml. The dilution is carried out using PBS. The resulting solution is then filtered through a 0.2 micron filter into a sterile container. After filtration, the solution is aliquoted into single doses of 1 ml and stored at −22° C. prior to use. The reagent is then ready for use.

Changes may be made in this procedure, such as for example by varying the concentration of the ammonium sulfate or switching to other reagents. Similarly, the dialysis cut-off need not be at 10,000 Daltons.

1. A method for identifying cancer-specific antibodies utilizing an immune checkpoint inhibition treatment in a subject who has cancer comprising the steps of:
a. obtaining cancer cells and/or their components, and normal cells and/or their components from the subject who has cancer;
b. administering the immune checkpoint inhibition treatment to the subject who has cancer;
c. allowing time for generation of an immune reaction against the cancer cells in the subject who has cancer;
d. obtaining a serum sample from subject who has cancer, the sample comprising any antibodies which may have been generated against the cancer cells;
e. optionally discontinuing the immune checkpoint inhibition treatment of the subject who has cancer;
f. removing from the serum of step d antibodies that bind to the normal cells and/or components by incubating the serum with a cell culture prepared of the normal cells and/or their components of step a, allowing sufficient time for antibodies/antigens binding and generation of antibodies/antigen complexes, and removing and keeping the supernatant;
g. selecting the antibodies that bind to the cancer cells and/or their components by incubating the supernatant of step f with a culture of the cancer cells and/or their components of step a, allowing sufficient time for antibodies/antigens binding and generation of antibodies/antigen complexes, keeping the culture comprising antibodies/antigen complexes for further analysis and discarding the supernatant;
h. extracting the antibody/antigen complexes from the culture of step g;
i. separating the antibodies and the antigens of the antibody/antigen complexes of step h using standard protocols;
j. analyzing the antigens of step i are using standard methods for antigen identification, such as mass spectrometry; and,
k. identifying sites or regions on the antigens of step j which are specific to the cancer cells or their components obtained from the subject who has cancer in step a.

2. A method of using the sites or regions identified in step k of claim 1 to produce a medication, wherein the medication may be antibodies specific to the sites or regions, or other therapeutic agents specific to the sites or regions, or to related sites or regions.

3. A method of treating a subject who has cancer, comprising administering to said subject a therapeutically effective amount of a pharmaceutical composition comprising the medication according to claim 2.

4. A method of treating a subject who has cancer, comprising administering to said subject a therapeutically effective amount of the isolated antibodies of step i of claim 1, wherein the antibodies are either unmodified or modified.

5. The method of claim 1, wherein the structure of at least one of an antigen's antibody/antigen attachment sites of the antibodies/antigens complexes of step i is determined, and the antigen's antibody/antigen attachment site is used as a treatment target for non-antibody based cancer treatments.

6. The method of claim 5, wherein the at least one antigen's antibody/antigen attachment site is used as a vaccine administered to an animal for creation of antibodies specific to the antigen's antibody/antigen attachment site.

7. A method of treating a subject who has cancer of claim 6 comprising administering to said subject a therapeutically effective amount of the antibodies specific to the antigen's antibody/antigen attachment site, wherein the antibodies are either unmodified or modified.

8. A method of detecting B cells which generate antibodies against the cancer cells in the subject who has cancer, comprising the steps of:
a. determining the amino acid sequence of the at least one of the antigen's antibody/antigen attachment sites of claim 5 and synthesizing and labeling a peptide comprising at least part of the said amino acids sequence, or labeling the at least one of the antigen's antibody/antigen attachment sites of claim 5; and,
b. incubating the labeled peptide or labeled at least one of the labeled antigen's antibody/antigen attachment sites of step b with the serum obtained from the subject who has cancer, and allowing time for specific binding between the labeled peptides and B cells.

9. The method of claim 8, wherein the type of label of the peptide of step a is selected from a group comprising radioactive isotopes, radiolabeled amino acid, and/or fluorescent amino acids.

10. The method of claim 9, wherein the labeled B cells are isolated, cell cultured, and induced to produce the cancer-specific antibodies.

11. A method of treating a subject who has cancer, comprising administering to said subject a therapeutically effective amount of the B cells of claim 10.

12. A method of treating a subject who has cancer, comprising administering to said subject a therapeutically effective amount of the antibodies, and/or their derivatives, produced by the B cells of claim 10.
